(19) United States
(12) Patent Application Publication
(10) Pub. No.: US 2009/0161384 A1
(43) Pub. Date: Jun. 25, 2009

(54) Beveled Tip Surgical Wide-Angle Illuminator

(76) Inventor: Ronald T. Smith, Irvine, CA (US)

Correspondence Address: ALCON, IP LEGAL, TB4-8, 6201 SOUTH FREEWAY, FORT WORTH, TX, 76134, US

(21) Appl. No.: 12/261,659

(22) Filed: Oct. 30, 2008

Related U.S. Application Data
(60) Provisional application No. 60/983,677, filed on Oct. 30, 2007.

(51) Int. Cl. A61B 1/06

(52) U.S. Cl. 362/572

(57) ABSTRACT

A beveled tip, wide-angle illuminator is disclosed, one embodiment comprising: a light source for providing a light beam; an optical cable, optically coupled to the light source for receiving and transmitting the light beam; a handpiece, operably coupled to the optical cable to receive the light beam; an optical fiber, operably coupled to the handpiece, wherein the optical fiber is optically coupled to the optical cable to receive and transmit the light beam; an optical element, optically coupled to a distal end of the optical fiber, for receiving the light beam and scattering the light beam to illuminate a surgical field, wherein the optical element comprises a beveled sapphire; and a beveled cannula, operably coupled to the handpiece, for housing and directing the optical fiber and the optical element. The optical element can be a small-gauge, diffusive sapphire element having a polished distal surface co-incident with the distal end of the cannula and a light refracting hemispherical surface facing the optical fiber.

### RELATED APPLICATIONS

This application claims priority to U.S. Patent Application No. 60/983,677 filed Oct. 30, 2007.

### TECHNICAL FIELD OF THE INVENTION

The present invention relates generally to surgical instrumentation. In particular, the present invention relates to surgical instruments for illuminating an area during eye surgery. Even more particularly, the present invention relates to a beveled tip, small gauge, wide-angle illuminator for illumination of a surgical field.

### BACKGROUND OF THE INVENTION

In ophthalmic surgery, and in particular in vitreo-retinal surgery, it is desirable to use a wide-angle surgical microscope system to view as large a portion of the retina as possible. Wide-angle objective lenses for such microscopic systems exist, but they require a wider illumination field than that provided by the cone of illumination of a typical fiber-optic probe. As a result, various technologies have been developed to increase the beam spreading of the relatively incoherent light provided by a fiber-optic illuminator. These known wide-angle illuminators can thus illuminate a larger portion of the retina as required by current wide-angle surgical microscope systems. Currently existing wide-angle illuminators, however, display several disadvantages.

One disadvantage exhibited by some prior art small gauge, wide-angle illuminators for ophthalmic surgery is that they are not able to provide a high luminous flux and high peak luminous intensity. Typically in these probes, wide angle beam spreading is achieved without sufficient intensity to permit a surgeon a clear and bright view of the surgical field.

Another disadvantage of currently available wide-angle illuminators is glare. Glare results when the source of the illumination is small and bright, and the user (e.g., an ophthalmic surgeon) has a direct line of sight to the small bright illumination source. Glare is unwanted stray radiation that provides no useful illumination, and either distracts an observer or obscures an object under observation. Glare can be corrected for in current wide-angle illuminators, but typically only by reducing the total illumination light flux, which reduces the amount of light available for observation by the surgeon. For example, the “bullet probe” manufactured by Alcon Laboratories, Inc., of Fort Worth, Tex., achieves wide-angle illumination by using a bullet-shaped fiber having a surface diffusive finish to scatter light emanating from the distal end of an optical fiber. To reduce glare, the bullet probe can use a geometric shield, which reduces the illumination angle by reducing the overall available light flux.

A further disadvantage of typical prior art wide-angle illuminators is that they are expensive to produce, a cost which is passed along to the surgeon and ultimately to the patient. As a result, prior art illuminators are typically not disposable and will require periodic maintenance and sterilization between surgical procedures.

Therefore, a need exists for a variable-intensity, wide-angle illuminator that can reduce or eliminate the problems of low luminous flux, low peak luminous intensity, glare, cost, efficiency and other problems associated with prior art wide-angle illuminators.

### BRIEF SUMMARY OF THE INVENTION

The embodiments of the beveled tip, wide-angle surgical illuminator of the present invention substantially meet these needs and others. One embodiment of this invention is a small-gauge, beveled tip, wide-angle illumination surgical system comprising: a light source for providing a light beam; an optical cable, optically coupled to the light source for receiving and transmitting the light beam; a handpiece, operably coupled to the optical cable to receive the light beam; an optical fiber, operably coupled to the handpiece, wherein the optical fiber is optically coupled to the optical cable to receive and transmit the light beam; an optical element, optically coupled to a distal end of the optical fiber, for receiving the light beam and scattering the light beam to illuminate a surgical field, wherein the optical element comprises a beveled sapphire; and a beveled cannula, operably coupled to the handpiece, for housing and directing the optical fiber and the optical element.

The optical element can be a small-gauge, optical-grade diffusive sapphire element having a polished flat circular surface co-incident with the distal end of the cannula and a light refracting hemispherical surface facing the optical fiber. The distal end of the cannula and the flat distal surface of the optical element can be beveled at a predetermined angle from the normal plane to the longitudinal axis of the cannula, as shown in FIGS. 2 and 3. The optical element can be sized, for example, for housing within a 19, 20 or 25 gauge cannula (e.g., about 0.75 mm to about 0.4 mm diameter optical element). Further, the cannula and the handpiece can be fabricated from biocompatible materials. The optical cable can comprise a first optical connector operably coupled to the light source and a second optical connector operably coupled to the handpiece (to optically couple the optical cable to the optical fiber housed within the handpiece and cannula). These connectors can be SMA optical fiber connectors. The optical element, optical fiber and optical cable (i.e., the optical fibers within the optical cable) should be of a compatible gauge so as to transmit the light beam from the light source to the surgical field. For example, all three elements could be of equal gauge.

Other embodiments of the present invention can include a method for wide-angle illumination of a surgical field using a beveled tip, wide-angle illuminator in accordance with the teachings of this invention, and a surgical handpiece embodiment of the beveled tip, wide-angle illuminator of the present invention for use in ophthalmic surgery. Embodiments of this invention can be implemented as a handpiece connected to a cannula, or other housing, including a fiber optic cable terminating in a diffusive optical element. Further, embodiments of this invention can be incorporated within a surgical machine or system for use in ophthalmic or other surgery. Other uses for a beveled tip, wide-angle illuminator designed in accordance with the teachings of this invention will be known to those having skill in the art.

### BRIEF DESCRIPTION OF THE SEVERAL VIEWS OF THE DRAWINGS

A more complete understanding of the present invention and the advantages thereof may be acquired by referring to the following description, taken in conjunction with the accompanying drawings, in which like reference numbers indicate like features and wherein:

FIG. 1 is a simplified diagram of one embodiment of a system for variable, wide-angle illumination in accordance with the teachings of this invention;

FIG. 2 is a more detailed diagram of a stem housing an embodiment of a diffusive optical element for wide-angle illumination in accordance with the teachings of this invention;

FIG. 3 is a diagram illustrating the use of an embodiment of a wide-angle illuminator of the present invention for ophthalmic surgery; and

FIG. 4 is a diagram illustrating an embodiment of an adjusting means 40 in accordance with the present invention.

### DETAILED DESCRIPTION OF THE INVENTION

Preferred embodiments of the present invention are illustrated in the FIGURES, like numerals being used to refer to like and corresponding parts of the various drawings.

The various embodiments of the present invention provide for a small gauge (e.g., 19, 20, or 25 gauge) optical fiber based endo-illuminator device for use in surgical procedures, such as in vitreo-retinal/posterior segment surgery. Embodiments of this invention can comprise a handpiece, such as the Alcon-Grieshaber Revolution-DSP™ handpiece sold by Alcon Laboratories, Inc., Fort Worth, Tex., connected to a small gauge cannula (e.g., 19, 20, or 25 gauge). The inner dimension of the cannula can be used to house one, or a plurality of, optical fibers terminating at a diffusive optical element in accordance with the teachings of this invention. Embodiments of the wide-angle illuminator can be configured for use in the general field of ophthalmic surgery. However, it is contemplated and it will be realized by those skilled in the art that the scope of the present invention is not limited to opthalmology, but may be applied generally to other areas of surgery where wide-angle illumination may be desired.

An embodiment of the beveled tip, wide-angle illuminator of this invention can comprise a light diffusive optical element fabricated of optical-grade sapphire, and a stem and a handpiece fabricated from biocompatible materials. Some embodiments can be made of polymeric materials such that the invasive portion of the wide-angle illuminator is a disposable surgical item. Unlike the prior art, each embodiment of the beveled tip, wide-angle illuminator of this invention can provide high optical transmission/high brightness with low optical losses and reduced glare. Embodiments of this invention fabricated from biocompatible polymeric materials can be integrated into a low cost, articulated handpiece mechanism, such that these embodiments can comprise an inexpensive disposable illuminator instrument.

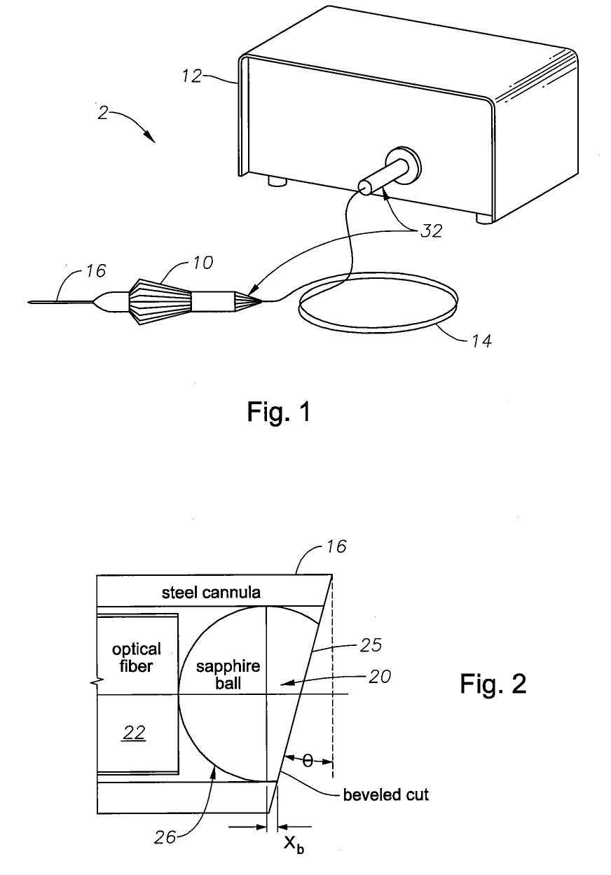

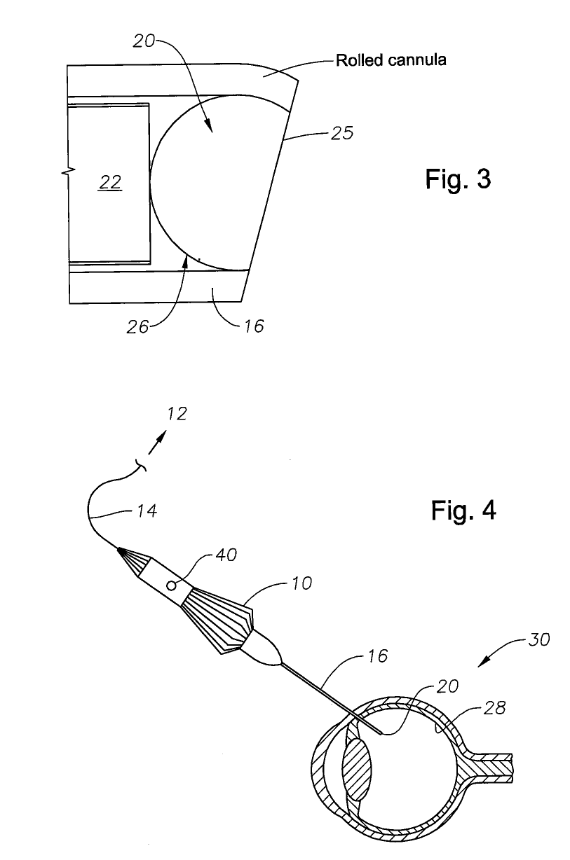

FIG. 1 is a simplified diagram of a surgical system 2 comprising a handpiece 10 for delivering a beam of relatively incoherent light from a light source 12 through cable 14 to a stem 16. Cable 14 can be any gauge fiber optic cable as known in the art, but is preferably a cable having 19, 20, or 25 gauge fiber. Further, cable 14 can comprise a single optical fiber or a plurality of optical fibers optically coupled to receive and transmit light from light source 12 to stem 16 through handpiece 10. Stem 16 is configured to house a diffusive optical element 20 at the distal end of stem 16, as is more clearly illustrated in FIG. 2. Coupling system 32 can comprise an optical fiber connector at each end of cable 14 to optically couple light source 12 to optical element 20 within handpiece 10, as discussed more fully below.

FIG. 2 is a magnified view of the distal end of stem 16. Stem 16 is shown housing fiber 22 and optical element 20. Stem 16 and optical element 20 are beveled at a predetermined angle θ from the normal plane to the longitudinal axis of stem 16. Optical element 20 is optically coupled to fiber 22, which is optically coupled to fiber optic cable 14. In some embodiments, fiber optic cable 14 can extend through the handpiece 10 and is optically coupled directly to optical element 20. For these embodiments, fiber 22 is not used. When implemented within handpiece 10, fiber 22 is of a gauge compatible with the gauge of fiber optic cable 14, such that it can receive and transmit light from fiber optic cable 14. Handpiece 10 can be any surgical handpiece as known in the art, such as the Revolution-DSP™ handpiece sold by Alcon Laboratories, Inc. of Fort Worth, Tex. Light source 12 can be a xenon light source, a halogen light source, or any other light source capable of delivering incoherent light through a fiber optic cable, as will be known to those having skill in the art. Stem 16 can be a small gauge cannula, preferably on the order of 19, 20, or 25 gauge, as known to those having skill in the art. Stem 16 can be stainless steel or a suitable biocompatible polymer (e.g., PEEK, polyimide, etc.) as known to those in the art.

The fiber optic cable 14 or fiber 22 housed within the stem 16 is operably coupled to the handpiece 10. Light source 12 can be optically coupled to handpiece 10 (i.e., to fiber 22) using, for example, standard SMA (Scale Manufacturers Association) optical fiber connectors at the proximal ends of fiber optic cable 14. This allows for the efficient coupling of light from the light source 12 through fiber optic cable 14 to the handpiece 10 and finally emanating from optical element 20 at the distal end of the stem 16. Light source 12 may comprise filters, as known to those skilled in the art, to reduce the damaging thermal effects of absorbed infrared radiation originating at the light source. The light source 12 filter(s) can be used to selectively illuminate a surgical field with different colors of light, such as to excite a surgical dye.

Fiber(s) 22 (and/or 14, depending on the embodiment) is/are terminated by optically coupling to optical element 20. Fiber 22 and optical element 20 can be optically coupled through direct contact. Optical element 20 can be an optical grade sapphire diffuser having a beveled semi-hemispherical shape. Optical element 20 can comprise a distal polished flat surface 25 at the distal end of stem 16 (i.e., facing out towards a surgical field) and a hemispherical surface 26 facing the distal end of fiber 22. The distal surface 25 of optical element 20 and the distal end of stem 16 are beveled at a predetermined angle θ from the normal plane to the longitudinal axis of stem 16. This predetermined angle θ can range up to about 45 degrees. Optical element 20 is sized for housing within stem 16 (e.g., a 19 to 30 gauge cannula). For example, optical element 20 can have a diameter of about 0.75 mm to about 0.4 mm. The flat surface 25 of optical element 20 can be co-incident with the open aperture at the distal end of stem 16.

Embodiments of the beveled tip, wide-angle illuminator of the present invention can be produced by removing a beveled section from the stem 16 and optical element 20. One method of fabrication can comprise locating a positioning pin inside stem 16 and then press fitting a sapphire ball optical element 20 inside the distal end of stem 16 so that it rests against the positioning pin. Optical grade adhesive can be used to bond the optical element 20 securely to stem 16 (such as in the embodiment of FIG. 2). A grinder, such as an angled grinder, can be used to grind away a desired beveled portion of the stem 16/optical element 20 assembly.

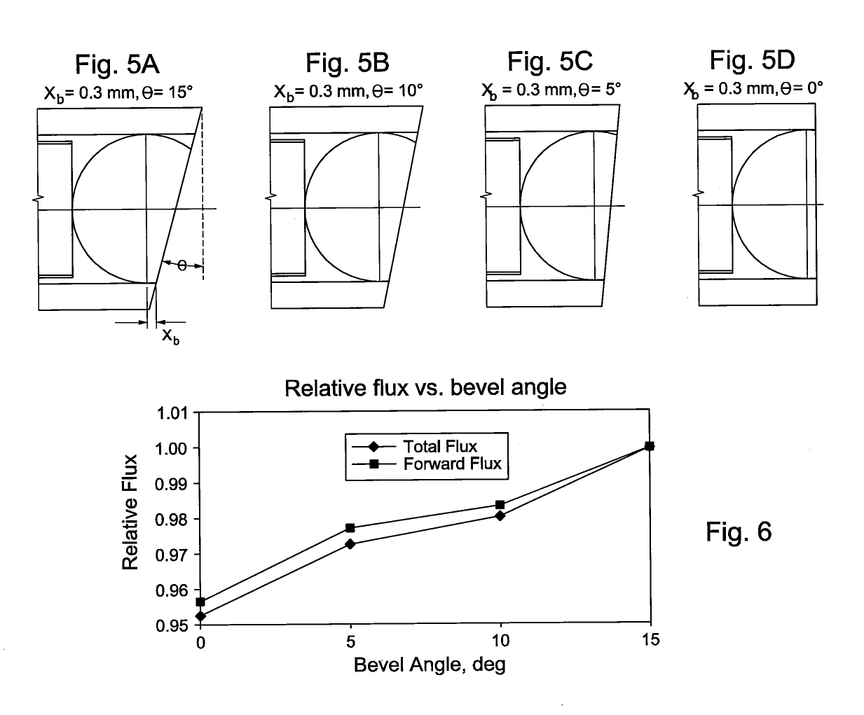

Alternatively, optical element 20 can be slip-fit into stem 16 and locked in place within stem 16 by a side roller to cause the distal end of stem 16 to roll over the edge of the distal surface 25 of optical element 20. A subsequent angled grinding step can be performed to bevel the stem 16/optical element 20 assembly to create an embodiment such as shown in FIG. 3. Optical grade adhesive can be used in any embodiment of the present invention to any spaces between the optical element 20 and stem 16 to prevent fluid entry into the space between the optical fiber 22 and optical element 20.

As shown in FIGS. 2 and 3, optical element 20 comprises a smooth semi-hemispherical sapphire. Optical element 20 can be a commercially available sapphire element known to those having skill in the art. The optical element 20, optically coupled to the distal end of the light carrying fiber 22, is housed inside stem 16. Stem 16 is itself operably coupled to the handpiece 10, which can be either a re-usable or a disposable handpiece 10.

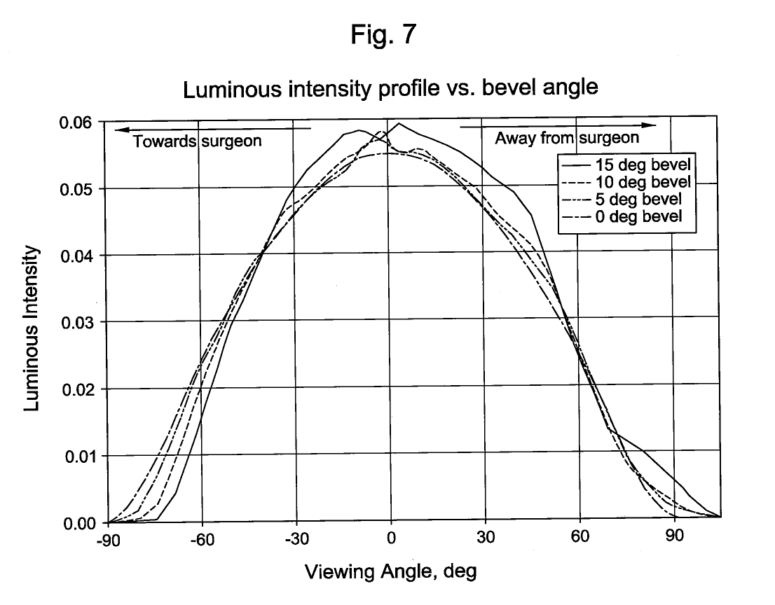

FIG. 4 illustrates the use of one embodiment of the beveled tip, wide-angle illuminator of this invention in an ophthalmic surgery. In operation, handpiece 10 delivers a beam of incoherent light through stem 16 (via fiber optic cable 14 and/or optical fiber 22) and through optical element 20 to illuminate a retina 28 of an eye 30. The collimated light delivered through handpiece 10 to optical element 20 is generated by light source 12 and delivered to illuminate the retina 28 by means of fiber optic cable 14 and coupling system 32. Optical element 20 spreads the light beam delivered from light source 12 over as large an area of the retina as, for example, a microscopic wide-angle objective lens permits a surgeon to see. The embodiments of the wide-angle illuminator of this invention can provide illumination angles up to about 160 degrees.

FIGS. 5a-5d, 6 and 7 illustrate calculated results of the effect of bevel angle on relative flux and luminous intensity for a beveled tip, wide-angle illuminator in accordance with the present invention. As can be seen from these FIGUREs, increasing the magnitude of the optical element 20 bevel angle results in increased flux, decreased glare towards a user (surgeon) and increased luminous intensity in the a direction away from the surgeon.

The embodiments of the beveled tip, wide-angle illuminator of this invention provide several advantages over the prior art, such as maximizing light transmission, a high angular spread (about 85-92 degrees FWHM angular bandwidth in saline solution), a high luminous flux (e.g., about 15-18 lumens for a 23 gauge probe) with an illuminator (light source 12) such as the Alcon High Brightness Illuminator, manufactured by Alcon Laboratories, Inc., of Irvine, Calif., a high peak luminous intensity, elimination of glare in the direction of the surgeon, and more light emitted in a direction away from the surgeon and onto a surgical field.

A traditional fiber-optic illuminator with a polished face will produce an included illumination angle that is a function of the numerical aperture (“NA”) of the fiber. NA defines the acceptance angle of entrance of the light from the light source into the fiber optic cable. Commonly, the fiber used for ophthalmic illumination applications has a typical NA of 0.5. This provides a calculated acceptance angle of 60 degrees in vacuo. Wide-angle viewing systems commonly used by ophthalmic surgeons typically have a viewing angle requirement greater than about 100 degrees in vivo. Thus, conventional fiber optic illuminators cannot provide a lighted field that matches the viewing system angle of visibility. The embodiments of the variable-intensity, wide-angle illuminator of this invention can provide an angle of illumination in excess of about 160 degrees (i.e., a range of illumination angles up to about 160 degrees).

Although the present invention has been described in detail herein with reference to the illustrated embodiments, it should be understood that the description is by way of example only and is not to be construed in a limiting sense. It is to be further understood, therefore, that numerous changes in the details of the embodiments of this invention and additional embodiments of this invention will be apparent to, and may be made by, persons of ordinary skill in the art having reference to this description. It is contemplated that all such changes and additional embodiments are within the spirit and true scope of this invention as claimed below. Thus, while the present invention has been described in particular reference to the general area of ophthalmic surgery, the teachings contained herein apply equally wherever it is desirous to provide wide-angle illumination, and where contact with a transparent fluid might normally interfere with the ability to obtain wide-angle illumination.

What is claimed is:

1. A small-gauge, wide-angle illuminator, comprising:
a handpiece, optically coupled to receive a light beam from a light source;
an optical fiber, operably coupled to the handpiece, wherein the optical fiber receives the light beam from the light source;
an optical element, optically coupled to a distal end of the optical fiber, for receiving the light beam and scattering the light beam to illuminate a surgical field, wherein the optical element comprises a beveled sapphire; and
a beveled cannula, operably coupled to the handpiece, for housing and directing the optical fiber and the optical element.

2. The small-gauge, wide-angle illuminator of claim 1, wherein the optical element is a small-gauge optical element having a distal surface co-incident with an open aperture of the cannula, and a hemispherical surface facing the optical fiber.

3. The small-gauge, wide-angle illuminator of claim 1, wherein the optical element is a 19, 20 or 25 gauge optical element.

4. The small-gauge, wide-angle illuminator of claim 1, wherein the cannula and the handpiece are fabricated from biocompatible materials.

5. The small-gauge, wide-angle illuminator of claim 1, wherein the optical fiber is optically coupled at the distal end to the optical element and at a proximal end to an optical cable, wherein the optical cable is operably coupled to the light source to transmit the light beam to the optical fiber, and wherein the optical cable comprises a first optical connector operably coupled to the light source and a second optical connector operably coupled to the handpiece.

6. The small-gauge, wide-angle illuminator of claim 5, wherein the optical cable gauge and the optical fiber gauge are equal.

7. The small-gauge, wide-angle illuminator of claim 5, wherein the optical element and the cannula are beveled at a predetermined angle from the normal plane to the longitudinal axis of the cannula.

8. The small-gauge, wide-angle illuminator of claim 7, wherein the distal end of the cannula is rolled over the distal surface edge of the optical element to operably couple the optical element to the cannula.

9. The small-gauge, wide-angle illuminator of claim 1, wherein the optical fiber gauge and the optical element gauge are equal.

10. The small-gauge, wide-angle illuminator of claim 14, wherein the angle of illumination is between 20 and about 160 degrees.

11. The small-gauge, wide-angle illuminator of claim 1, wherein the light beam comprises a beam of incoherent light.

12. The small-gauge, wide-angle illuminator of claim 1, wherein the light source is a xenon light source.

13. The small-gauge, wide-angle illuminator of claim 1, wherein the optical element is about 2 millimeters long.
